(19) United States
(12) Patent Application Publication
(10) Pub. No.: US 2019/0168012 A1
(43) Pub. Date: Jun. 6, 2019

(54) MAGNETIC STIMULATION COIL ARRANGEMENT

(71) Applicant: The Magstim Company Limited, Whitland (GB)

(72) Inventor: Matthew Biginton, Newcastle (GB)

(73) Assignee: The Magstim Company Limited, Whitland (GB)

(21) Appl. No.: 16/171,532

(22) Filed: Oct. 26, 2018

Related U.S. Application Data
(63) Continuation of application No. PCT/GB2017/051190, filed on Apr. 28, 2017.

(30) Foreign Application Priority Data
Apr. 28, 2016 (GB) 1607384.3

(51) Int. Cl. A61N 2/02

(52) U.S. Cl. CPC A61N 2/02

(57) ABSTRACT

The present invention relates to a magnetic stimulation (MS) coil arrangement which utilises the effect of the position of a ferromagnetic material to enhance the magnetic field on the patient side of the coil and reduce acoustic noise associated with the addition of ferromagnetic material. The present invention comprises a magnetic stimulation coil arrangement comprising one or more coil windings formed from an elongate conductive element and having a forward side for presentation to a patient and a rearward side, the magnetic stimulation coil arrangement further comprising a distortion arrangement for distorting a magnetic field produced by the one or more coils positioned adjacent to the rearward side, the distortion arrangement having a plurality of ferromagnetic components and a carrier for carrying the ferromagnetic components, the ferromagnetic components being spaced from one another by the carrier.

### CROSS-REFERENCE TO RELATED APPLICATION

The present application is a continuation of PCT Application No. PCT/GB2017/051190, filed Apr. 28, 2017, which claims priority to United Kingdom application no. 1607384.3, filed Apr. 28, 2016, the contents of each of which are hereby incorporated by reference in their entirety for all purposes.

The present invention relates to a Magnetic Stimulation (MS) coil arrangement which utilises the effect of the positioning of a ferromagnetic material to enhance the magnetic field on the patient side of the coil and reduce acoustic noise associated with the addition of ferromagnetic material.

MS coil arrangements include an apparatus for transmitting at least one pulse of current through a generally circular coil or a figure of eight coil arrangement having one or more windings, each having a plurality of turns. MS coils can be produced in a variety of shapes sizes and arrangements. Typical stimulating coils comprise an elongate conductive element wound into a coil having a plurality of turns whereby the turns are insulated from each other. When a current is passed through the wound elongate conductive element the magnetic field that is generated transfers to a patient to give a therapeutic effect or to research how various parts of the brain or body operate.

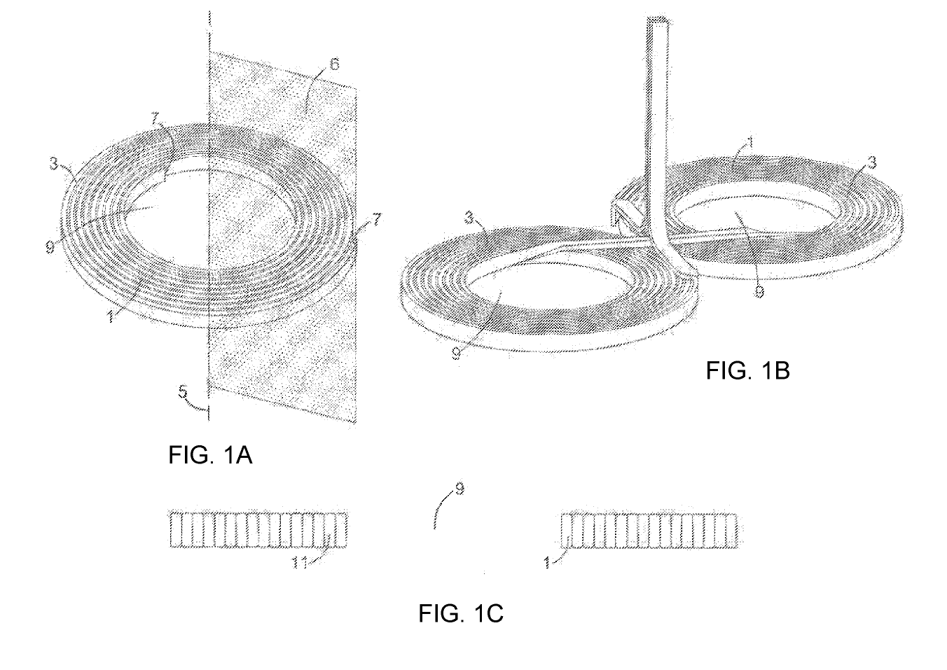

FIG. 1A is a three dimensional schematic representation of a circular MS coil (1) comprising a coil winding (3) having a plurality of turns. The centre (symmetry) line of the coil is marked with a dashed line (5). The coil (1) is connected to a stimulator (not shown) via the two ends of the coil winding (7). FIG. 1B shows a double TMS coil (or Figure of eight TMS coil) comprising two coil windings (3) to show the variety of different coils available. Each winding (3) typically has an opening (or hole) (9) at its centre. FIG. 1C shows a cross section of the turns of a circular winding showing the individual turns (11).

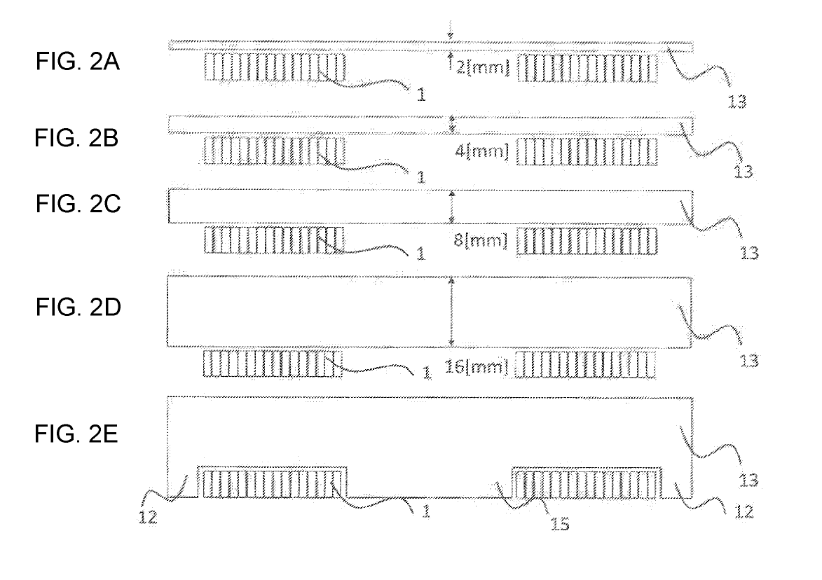

FIGS. 2A-2E are cross sectional views of the circular MS coil (1) shown in FIGS. 1A and 1C with varying sized and shaped ferromagnetic slabs (discs in this example) (13) placed on the operator (rearward) side of the coil. FIGS. 2A, 2B, 2C and 2D show 2 mm, 4 mm, 8 mm and 16 mm thick slabs respectively placed on the back of the circular coil. In FIG. 2E the ferromagnetic material has been extended (12) into the aperture (9) of the winding and around the peripheral edges of the coil (1).

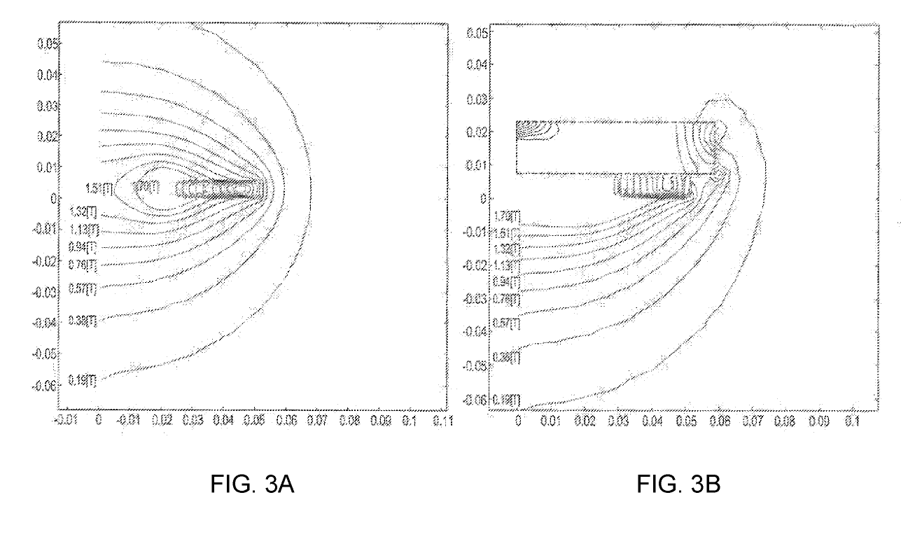

By positioning a ferromagnetic material (13) behind the coil (1) the magnetic field in comparison to the circular coil (1) alone as (illustrated by finite element modelling in FIG. 3B) is distorted and is now asymmetric about the plane of the coil. The reluctance of the material on the rearward side of the coil (1) is now significantly lower and as a result most of the energy delivered to the coil is now stored in the volume on the forward side of the coil. This means that for the same applied energy the magnetic field strength is higher on the forward side of the coil (in the patient's tissue). Alternatively, the same magnetic field strength may be supplied to the patient whilst using less energy in comparison to the coil without the ferromagnetic material. This means that not only less energy is used to drive the coil but less joule heating occurs in the coil windings and can increase the amount of stimulations the coil can perform before overheating.

FIGS. 3A and 3B are a comparison of the finite element modelled magnetic field contour lines around the circular MS coil (1) shown in FIG. 1A and the circular MS coil shown in FIG. 1D with a 16 mm thick disc of ferromagnetic material placed adjacent its rearward side. In both cases the field lines are shown on the symmetry plane (5) illustrated in FIG. 1A. As can be clearly seen the field strength on the patient side (forward side) of the coil is increased by a significant amount. For clarity a dotted line outlines the ferromagnetic disc.

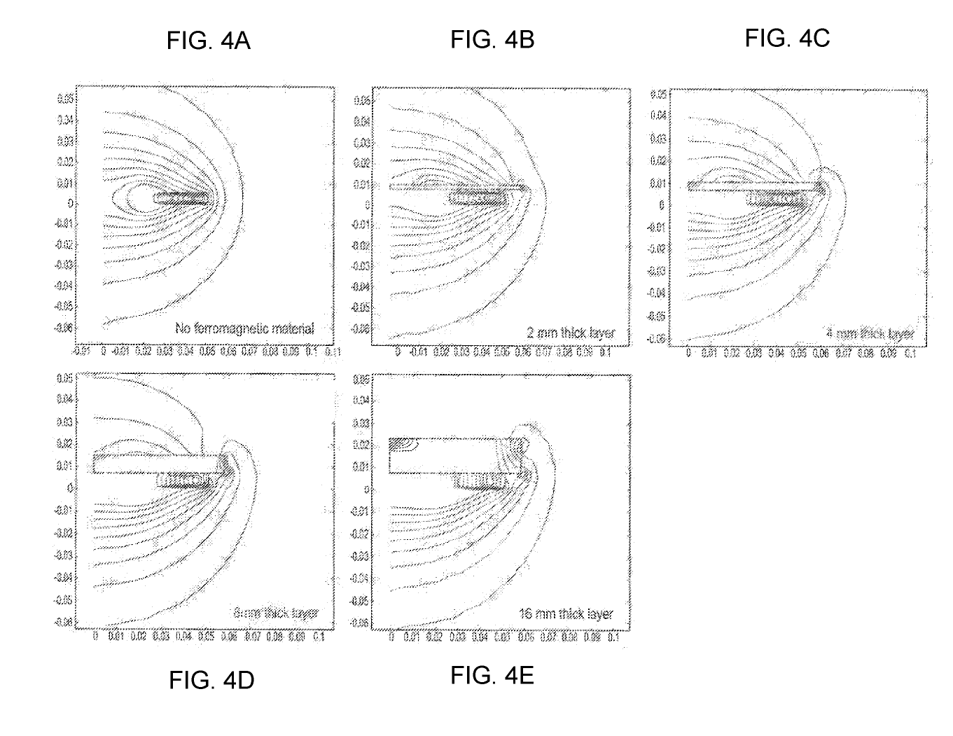

FIGS. 4A-4E illustrate the effect of increasing disc thickness of the ferromagnetic material (13) from 2 mm to 16 mm. As can be seen from the modelled contour maps the field strength on the patient side of the coil (1) is higher in comparison to the circular coil when the 2 mm disc is placed on the rearward side of the coil presented in FIG. 4A. Having a 4 mm layer increases the field strength further (b), 8 mm further again (c) and 16 mm further still (d). In the case of the 16 mm layer the increase has nearly leveled off and any further increase in layer thickness will have only a marginal effect on field enhancement.

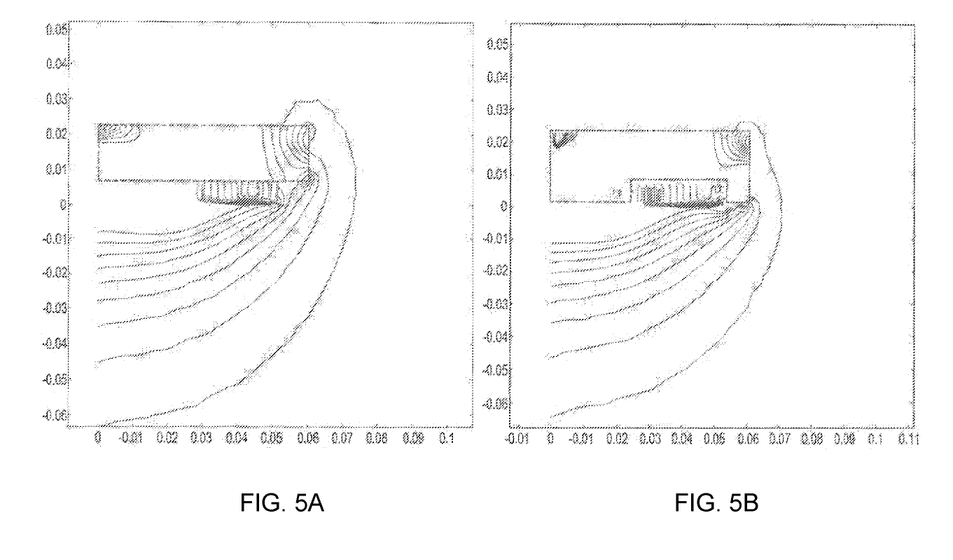

FIGS. 5A and 5B compare the field contour lines for a thick 16 mm disc of ferromagnetic material (shown in FIG. 2D) and the same disc albeit with the ferromagnetic material extending down into the coil winding aperture (9) and around the periphery of the coil (1). Clearly there is a greater increase in field strength with the extended sections (15, 12) of ferromagnetic material.

Finite element results shown in FIGS. 3A, 3B, 4A-4E, 5A, and 5B are modelled on a 2D plane in a 2D asymmetric model in COMSOL multi-physics which predicts the solutions assuming azimuthal symmetry.

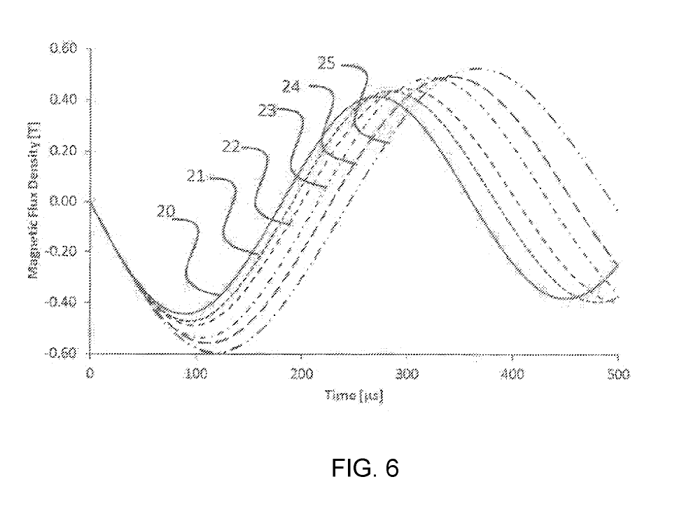

FIG. 6 illustrates the magnetic field pulse waveforms from finite element modelling as recorded 2 cm beneath the turns of the MS coil winding. Reference numerals 20, 21, 22, 23, 24 and 25 show the waveforms for the field distortion arrangements shown in FIGS. 2A, 2B, 2C, 2D and 2E respectively again illustrating that a thicker ferromagnetic materials on the back of the coil (1) produces an increased field strength at the front of the coil (1). In addition the plot clearly shows the benefit of extending the ferromagnetic material into the aperture (9) of the coil (1) around the peripheral edges.

There are problems associated with positioning a ferromagnetic material adjacent the coil at the operator's side. A solid ferromagnetic (metal) plate for example generates significant Eddy currents and quickly heats up. This significantly limits the number of pulses of current that may be supplied through the coil before the plate becomes too hot. In addition these eddy currents tend to reduce the benefits from placing a ferromagnetic metal around the coil as described in patent WO2016/005719.

An alternative ferromagnetic material has previously been utilised comprising grains of iron of approximately 0.1 mm in diameter, each electrically insulated from an adjacent grain by a very thin inorganic insulation material. These insulated grains are then sintered and subsequently cut to the desired shape. A problem that exists with the provision of sintered encapsulated iron grains is this material, as with many other ferromagnetic materials may become saturated in particular for the case of smaller MS coils which tend to produce much higher magnetic flux densities than their larger counterparts. This means an increase in the applied external magnetic field cannot significantly increase the magnetisation of the material further so the total magnetic flux density levels off. This means that the magnetic treatment field is not as high as intended or selected as the ferromagnetic material is no longer acting as such, and thus has less effect upon the magnetic field produced. Furthermore, upon saturation the temperature of the material may increase rapidly thus meaning that operation of the magnetic stimulation arrangement must be paused. This is particularly relevant for magnetic stimulation of a patient whereby the desirable magnetic flux density may typically be as high as three Tesla at some locations near the coil.

A second problem that exists with placing electrically insulated grains as described above and solid plates of ferromagnetic material on the operator side of a TMS coil is that they tend to enhance not only the magnetic field on the patient side of the coil but also the noise generated from the magnetic stimulation coil arrangement. This is considered to be an adverse effect as it is typical for patients to have to wear ear protection with standard TMS coils and additional noise is therefore undesirable for both patient and operator.

The present invention provides an improved solution which is easily implemented, cheap, effective and in particular aids in reducing the noise produced by the magnetic stimulation coil arrangement solving the second problem with the prior art.

According to the present invention there is a magnetic stimulation coil arrangement for use in apparatus for the magnetic stimulation of tissue, the magnetic stimulation coil arrangement comprising one or more coil windings formed from an elongate conductive element and having a forward side for presentation to a patient and a rearward side, the magnetic stimulation coil arrangement further comprising a distortion arrangement for distorting a magnetic field produced by the one or more windings positioned adjacent to the rearward side, the distortion arrangement having a plurality of ferromagnetic components and a carrier for carrying the ferromagnetic components, the ferromagnetic components being spaced from one another by the carrier.

The provision of the ferromagnetic components being spaced from one another and beneficially electrically insulated from one another causes the distortion arrangement to have a lower permeability due to the provision of the spacings (effectively air gaps). This typically allows the ferromagnetic components to withstand a higher applied external magnetic field strength before saturating. The present invention typically will have a lower permeability than either solid metal or encapsulated grains of iron in the known material “Somaloy”, which may saturate at a too low a flux density and which in some cases can lead to overheating. This leads to significant downtime as cooling of the distortion arrangement is required between pulses.

The ferromagnetic components are retained in the carrier through being encapsulated and/or embedded in the carrier. The carrier is a solid at room temperature and pressure.

The carrier is beneficially a matrix in which the ferromagnetic components are dispersed. The ferromagnetic components are beneficially randomly dispersed in the matrix. Dispersion of ferromagnetic components in the matrix reduces manufacturing costs whilst also providing the beneficial properties of the distortion arrangement.

The carrier is beneficially an electrical insulator and may comprise a polymer or ceramic.

The term component means a manufactured or formed object. Examples of components are ball bearings, nuts, bolts, discs, rods, cubes or parts of such objects. This improves ease and cost effectiveness of manufacture of the distortion arrangement.

The ferromagnetic components are beneficially dispersed within the carrier such that the distortion arrangement saturates at an applied field generating a flux density of greater than 1.5 Tesla.

The distortion arrangement is preferably positioned adjacent to and preferably parallel to the rearward side.

The distortion arrangement preferably comprises an array of ferromagnetic components. The ferromagnetic components are beneficially regularly spaced within the array for ease and repeatable manufacturing. However they may be randomly spaced. The ferromagnetic components are beneficially embedded or encapsulated in the carrier. The array may be an ordered array, however for ease of manufacture the ferromagnetic components in the array may be randomly positioned and oriented. This may be achieved by simply mixing the ferromagnetic components in the carrier provided in flowable form and allowing the carrier to set in a mould of the desired shape.

The distortion arrangement preferably includes a plurality of ferromagnetic components having matching dimensions for ease of manufacture. However it is appreciated that non matching dimensions or shapes will have a similar effect. The ferromagnetic components are beneficially manufactured or formed components. A plurality of the ferromagnetic components preferably have the same dimensions. The plurality of ferromagnetic components are preferably substantially identical. The provision of spherical ferromagnetic components provides a cheap and readily available ferromagnetic component that may be easily positioned in a carrier.

The maximum dimension of the ferromagnetic components is preferably between 0.045 and 1 cm. The maximum dimension is preferably less than 3 cm as it will be appreciated that as the size of ferromagnetic component significantly increases and progresses beyond 2 cm then more significant eddy currents are induced in the ferromagnetic components thus leading to the heating of the distortion arrangement. A lower limit dimension of approximately 0.045 cm is beneficial as lower than this means that it is difficult to maintain sufficient separation gap between ferromagnetic components leading to saturation at a too low a value of externally applied magnetic field strength thus reducing or removing the beneficial properties of the distortion arrangement.

The array may comprise a single layer of ferromagnetic components.

The array may comprise multiple layers of ferromagnetic components.

The carrier between the ferromagnetic components is preferably chosen to dampen the sound produced from the distortion arrangement and mechanically hold the ferromagnetic components in situ.

Typically the average spacing between ferromagnetic components may be 1/10 the size of the ferromagnetic components. However it is appreciated that by varying the gap size between the ferromagnetic components will change the effective permeability of the distortion arrangement. Therefore this average gap size could be as large as 3/1. The average gap may be as small as 1/25 in the case of 0.045 cm ferromagnetic component and 1/500 in the case of the largest ferromagnetic components (3 cm). It will be appreciated that there may be some touching of ferromagnetic components, particularly when mixed in size and/or shape however it is preferable that this is minimised.

The array may comprise a three dimensional array and the ferromagnetic components may be randomly spaced in the array. By providing a randomly spaced plurality of ferromagnetic components in the carrier the ferromagnetic components may be simply mixed in the carrier and formed in a mould providing the distortion arrangement.

The distortion arrangement may be arranged to correspond to the shape of the rearward side of the one or more coil windings. The distortion arrangement may comprise one or more apertures therein, each of the one or more apertures arranged to be aligned with corresponding apertures found radially inwardly of the elongate conductive element forming the one or more windings.

The or each of the coil winding preferably comprises a radially inner aperture, and the distortion arrangement may comprise a projection arranged to extend into the aperture. This has been found to further improve the effectiveness of the distortion arrangement.

The rearward side of the one or more windings comprises a peripheral edge, and the distortion arrangement may comprise a lip that extends around at least a portion of the peripheral edge. This again further improves the effectiveness of the distortion arrangement.

The ratio of the volume of ferromagnetic components to carrier material in the distortion arrangement is preferably less than 1.5:1, and preferably less than 1:1.

According to a second aspect of the present invention there is a magnetic stimulation coil arrangement for use in apparatus for the magnetic stimulation of tissue, the magnetic stimulation coil arrangement comprising one or more coil windings formed from an elongate conductive element and having a forward side for presentation to a patient and a rearward side, the magnetic stimulation coil arrangement further comprising a distortion arrangement for distorting a magnetic field produced by the one or more coils positioned adjacent to the rearward side, the distortion arrangement having a plurality of ferromagnetic particles and a carrier for carrying the ferromagnetic particles, the ferromagnetic particles being spaced from one another by the carrier wherein the ratio volume of ferromagnetic particles to carrier material is less than 1.5:1.

This provides the advantage that noise is effectively attenuated whilst most of the energy delivered to the coil is now stored in the volume on the forward side of the coil.

The ratio of the volume of ferromagnetic particles to carrier material is preferably less than 1:1.

The ferromagnetic particles may for example comprise iron filings.

Preferred features of the first aspect should also be understood as being preferred features of the second aspect.

The present invention will now be described by way of example only with reference to the accompanying drawings in which:

FIG. 1A is a three dimensional representation of a magnetic stimulation coil winding, FIG. 1B is a double TMS coil winding and FIG. 1C is a cross-section of a single magnetic stimulation coil winding.

FIGS. 2A-2E are schematic representations of the magnetic simulation coil winding as presented in FIGS. 1A and 1C with varying sized and shaped ferromagnetic disks placed on the rearward side of the coil winding.

FIGS. 3A and 3B are schematic representations of finite element modelled magnetic field contour lines around the magnetic stimulation coil winding shown in FIG. 1A and the magnetic stimulation coil winding as shown in FIG. 1D for comparative purposes.

FIGS. 4A-4E are illustrations of the finite elements modelled magnetic field contour lines produced by increase of the thickness of the ferromagnetic material.

FIGS. 5A and 5B are a comparison of the finite element modelled magnetic field contour lines for a 16 mm disk of ferromagnetic material in comparison to the same disk having additional formations.

FIG. 6 is a graphical representation of the magnetic field pulse wave forms from finite element modelling as recorded beneath the turns of MS coil winding of the distortion arrangements as presented in FIGS. 2A-2E respectively.

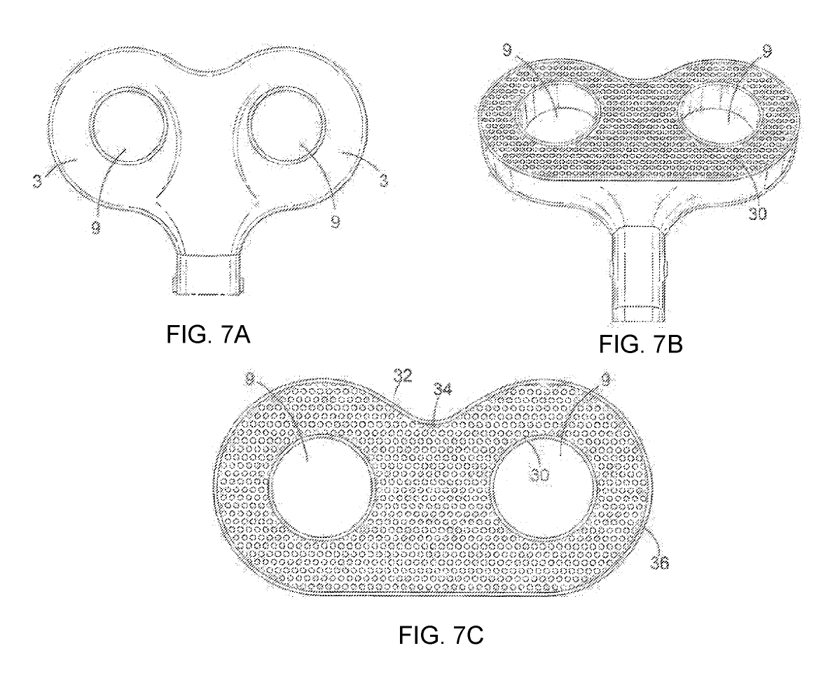

FIGS. 7A-7C is a representation of an exemplary embodiment of the present invention where FIG. 7A is a representation of the patients side of a double coil winding, FIG. 7B is the representation of the rearward or non-patient side of a magnetic simulation coil arrangement including a distortion arrangement according to an exemplary embodiment of the present invention and FIG. 7C is a schematic representation of a distortion arrangement according to an exemplary embodiment.

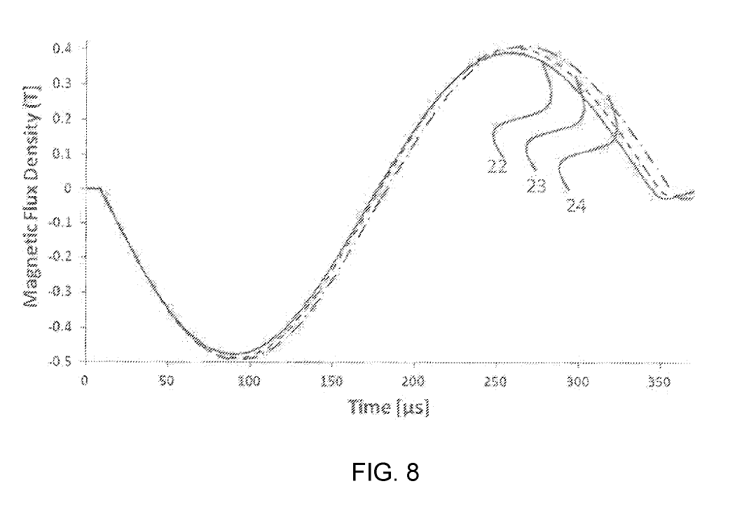

FIG. 8 is a graphical representation of the measured magnetic field on the forward or patient side of the magnetic stimulation coil windings for the exemplary embodiment as shown in FIGS. 7A-7C.

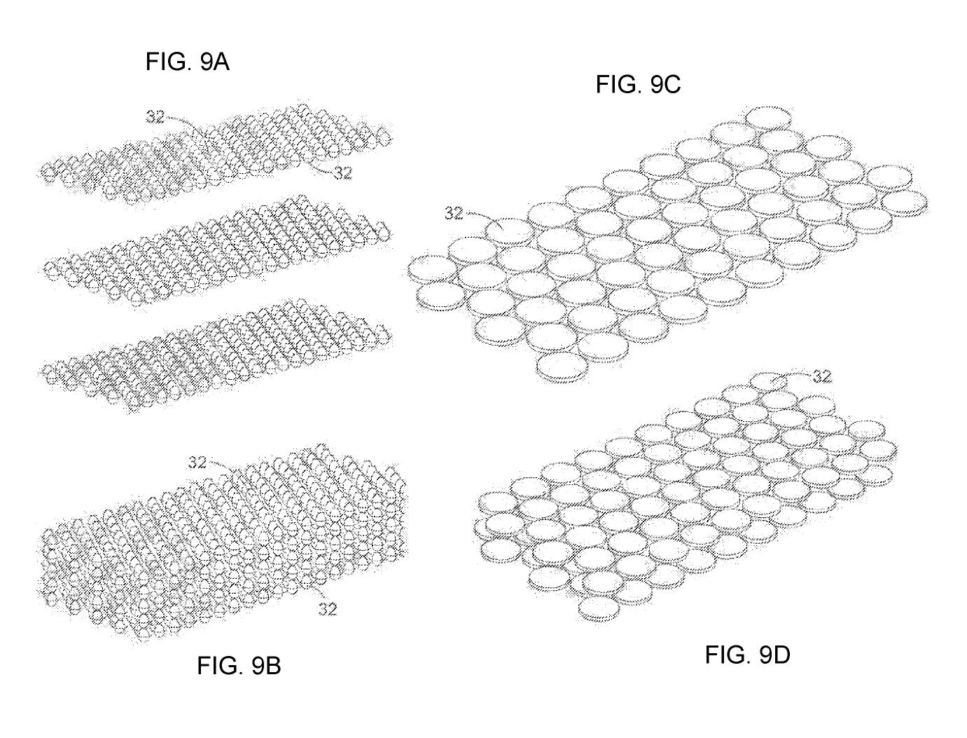

FIGS. 9A-9D are exemplary embodiments of ferromagnetic components for distortion arrangements according to an exemplary embodiment.

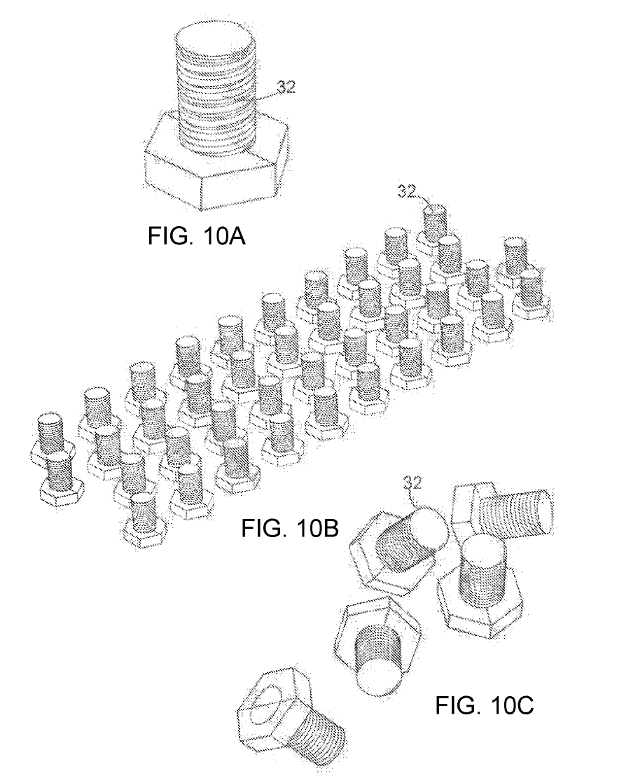

FIG. 10A-10C is a schematic representation of further ferromagnetic components for use in distortion arrangements according to an exemplary embodiment.

Referring to FIG. 7A the forward side of the magnetic stimulation coil arrangement is represented for presenting to a patient. Represented is a double coil winding made up of two windings (3) covered to ensure generated heat does not transfer to the patient. At the rearward side as presented in FIG. 7B is a distortion arrangement (30) secured to the magnetic stimulation coil windings (3). As better presented in FIG. 7C, the distortion arrangement comprises a plurality of ferromagnetic components (32), shown in a carrier material (34). The carrier material (34) acts to both increase the external field strength for which the ferromagnetic component array saturates (due to the gaps) and dampens noise from the distortion arrangement. In the embodiment presented the ferromagnetic components (32) are encapsulated in the carrier material (34). In the exemplary embodiment a hexagonal array of 3 mm ferromagnetic ball bearings have been provided in an array stacked two layers high. The ball bearings are separated by the carrier material (34) comprising a carrier resin and are held in place in a tray (36) which defines the outer peripheral edge and also defines the apertures (9) in the distortion arrangement (30). In this exemplary embodiment apertures (9) are presented and are aligned with the corresponding apertures in the middle of the magnetic stimulation coil windings (3). It will be appreciated, however, that the distortion arrangement (30) may be configured to cover these apertures and even more beneficially project into the apertures for increased efficiency at the forward side of the MS coil arrangement.

It should be noted that in the exemplary embodiment the ferromagnetic components (32) are shown as spheres or ball bearings embedded in the carrier (34). However alternative shapes of ferromagnetic components may be utilised such as discs for example shown in FIGS. 9A-9D square, hexagons, triangle or any other shape. It will also be appreciated that irregular shapes may be utilised as shown in FIGS. 10A-10C, however for consistency of operation the ferromagnetic components (32) are preferably of consistent shape. The maximum dimension of the ferromagnetic components is preferably less than 2 cm or ⅕ the diameter of the MS coil, and is preferably in the range of 0.045 cm to 1 cm. Such size of ferromagnetic components combined with the carrier (34) provides a distortion arrangement that can withstand a higher applied magnetic field strength before the magnetic flux density in the distortion arrangement causes the distortion arrangement to reach saturation point.

Referring now to FIG. 8 there is a graphical representation of the magnetic field output over time associated with a coil with no distortion arrangement measured adjacent the forward side (as presented in curve 22) and the same measurement over time associated with a coil with the distortion arrangement comprising two full layers of ball bearings (32) and a partially filled layer positioned the rearward side (as presented in curve 24). The graph clearly shows the increase in maximum magnetic field output associated with the coil with the distortion arrangement positioned adjacent the forward side. As the number of layer of ferromagnetic components increases so does the enhancement of the field.

A further important feature of the graph presented in FIG. 8 is the difference in time period associated with operation of the coil with and without a distortion arrangement. As described above, it is important that the distortion arrangement saturates at a magnetic field strength typically associated with the use of magnetic stimulation coils, and particularly transcranial magnetic stimulation coils (TMS coils). For this reason it is important that the distortion arrangement does not saturate at high applied magnetic fields. This can be checked as follows. The inductance of the coil windings (3) can be measured both with and without the distortion arrangement present at a low field strength known to be well below the saturation level. This measurement shows that the inductance of the coil increases by approximately 0.8 microH. There is also an associated change in the time period. Thus, if the distortion arrangement is not saturating at a higher applied field, then the time period will be expected to be the same, as if the distortion arrangement was saturating the time period may be different to the expected change from the increased inductance and show signs of distortion from an attenuated sine wave. The reason for this is that in the event of saturation of the distortion arrangement, the distortion arrangement will stop acting as a ferromagnetic material. The distortion arrangement according to the present invention has been shown to maintain ferromagnetic properties and thus not saturate for a typical MS coil arrangement operated at full power. In addition the waveforms are clearly not distorted from the expected attenuated sine waveform which also suggests the ferromagnetic material is not being saturated.

An advantage associated with the provision of the array of ferromagnetic components embedded or encapsulated within the carrier (34) is the lower temperature rise during operation in comparison to a solid ferromagnetic plate. This is achieved through the relative spacing between the ferromagnetic components (32) in the carrier 3(4). The reduction in temperature rise within the distortion arrangement is achieved due to eddy currents being limited to only being induced within each ferromagnetic component (32) rather than through the distortion arrangement as a whole. As such, the carrier material between each ferromagnetic component (32) acts to break up and hence minimise the eddy currents.

It is also noted that extending the distortion arrangement into the centres of the coil windings and around the periphery of the coil windings may also be advantageous. This is shown in FIG. 2E and also enhances the field.

The present invention may be manufactured with relative ease. The ferromagnetic components may be pressed into an insulating polymeric carrier material 14, which is typically polymeric but may also be for example ceramic. A high thermally conducting potting compound may be used as the carrier material to carry heat away from the coils in addition to its other functions. The ferromagnetic components may be mixed into a fluidic carrier material which subsequently solidifies to form a flexible or rigid body. The ferromagnetic components are then dispersed in a carrier material matrix.

The present invention may be implemented into a non-planar distortion arrangement. This means that the distortion arrangement can be formed to accommodate the contours of a selected coil. For example, in a figure of eight coil comprising first and second coils each of the coils may be tilted relative to the other coil. A distortion arrangement may therefore be formed with relative ease to accommodate such a configuration due to the ease of working with ferromagnetic components having a maximum dimension of between 0.1 and 3 cm, and preferably in the range 0.1 to 1 cm, and even more preferably in the range between 0.4 and 1 cm. Such scale of ferromagnetic components lend themselves to being moulded with a carrier material. The carrier material may comprise a potting compound, flexible rubber or other suitable non-metallic material.

The shape of the carrier material may be adjusted dependent upon the coil to be used. The shape preferably generally matches the shape of the coil.

Referring to FIGS. 9A to 9D alternative configurations of the ferromagnetic components in the distortion arrangement (30) are presented. In FIG. 9A layers of ferromagnetic components (32) are presented spaced apart for clarity purposes and in FIG. 9B as show in a tightly packed array of five layers deep presented as an exemplary embodiment only as ball bearings in a hexagonal array. FIG. 9C is a presentation of ferromagnetic components in the form of ferromagnetic disks (32) and FIG. 9D presents ferromagnetic disks (32) two layers deep. It will be appreciated that in any embodiment the ferromagnetic components (32) are embedded, encapsulated or otherwise retained in a carrier material.

The efficacy of different shapes of ferromagnetic components (32) has been tested, and it has been found that shapes other than spherical achieve similar effects provided with the carrier material (34). For example, a distortion arrangement comprising a plurality of steel bolts as shown in FIGS. 10A-10C potted in a thermosetting plastic or silicone rubber gel as the carrier (32) achieves saturation at a higher applied magnetic field.

In an embodiment according to another aspect of the invention it is possible to use, for example, iron filings or iron particles as a ferromagnetic material. However, the effectiveness of noise reduction must still be achieved and as such there must be sufficient quantity of carrier material in order to accommodate or attenuate the noise generated. For this reason the ratio of the volume of ferromagnetic particles to carrier material is less than 1.5:1, preferably less than 1:1.

Again, the particles can be mixed into a fluid carrier which is subsequently poured into a mould and solidified to form the distortion arrangement (30).

Aspects of the present invention enable enhancement of the magnetic field on the patient or forward side of the coil when placed on or adjacent to the rearward side. The increase in magnetic field strength on the patient side has been shown to be around 10%, meaning that the power supplied to the coil may now be lowered by 10% to achieve the same magnetic field output to the patient. This is achieved without the distortion arrangement saturating, and without the distortion arrangement overheating.

Aspects of the present invention have been described by way of example only and it will be appreciated by the skilled addressee that modifications and variations may be made without departing from the scope of protection afforded by the appended claims.

1. A magnetic stimulation coil arrangement for use in apparatus for the magnetic stimulation of tissue, the magnetic stimulation coil arrangement comprising one or more coil windings formed from an elongate conductive element and having a forward side for presentation to a patient and a rearward side, the magnetic stimulation coil arrangement further comprising a distortion arrangement for distorting a magnetic field produced by the one or more coils positioned adjacent to the rearward side, the distortion arrangement having a plurality of ferromagnetic components and a carrier for carrying the ferromagnetic components, the ferromagnetic components being spaced from one another by the carrier.

2. A magnetic stimulation coil arrangement according to claim 1 wherein the carrier is a matrix in which the ferromagnetic components are dispersed.

3. A magnetic stimulation coil arrangement according to claim 2 wherein the ferromagnetic components are randomly dispersed in the matrix.

4. A magnetic stimulation coil arrangement according to claim 1 wherein the carrier is an electrical insulator.

5. A magnetic stimulation coil arrangement according to claim 1 wherein the distortion arrangement comprises an array of ferromagnetic components.

6. A magnetic stimulation coil arrangement according to claim 5 wherein the array comprises a single layer of ferromagnetic components.

7. A magnetic stimulation coil arrangement according to claim 6 wherein the array comprises a three dimensional array and where the ferromagnetic components are randomly spaced in the array.

8. A magnetic stimulation coil arrangement according to claim 5 wherein the array comprises multiple layers of ferromagnetic components.

9. A magnetic stimulation coil arrangement according to claim 8 wherein the array comprises a three dimensional array and where the ferromagnetic components are randomly spaced in the array.

10. A magnetic stimulation coil arrangement according to claim 1 wherein the ferromagnetic components are embedded or encapsulated in the carrier.

11. A magnetic stimulation coil arrangement according to claim 1 wherein the ferromagnetic components include a plurality of ferromagnetic components having matching dimensions.

12. A magnetic stimulation coil arrangement according to claim 1 wherein the maximum dimension of the plurality of ferromagnetic components is 3 cm.

13. A magnetic stimulation coil arrangement according to claim 1 wherein the maximum dimension of the plurality of ferromagnetic components is between 0.045 and 3 cm.

14. A magnetic stimulation coil arrangement according to claim 1 wherein the ratio of the volume of ferromagnetic components to the carrier material in the distortion arrangement is less than 1.5:1 and preferably less than 1:1.

15. A magnetic stimulation coil arrangement according to claim 1 wherein the distortion arrangement is arranged to correspond to the shape of the rearward side of the one or more coil windings.

16. A magnetic stimulation coil arrangement according to claim 1 wherein the distortion arrangement comprises one or more apertures therein, each of the one or more apertures arranged to be aligned with corresponding one or more apertures found radially inwardly of the elongate conductive element forming the one or more windings.

17. A magnetic stimulation coil arrangement according to claim 1 wherein the or each of the coil windings comprises a radially inner aperture, and the distortion arrangement comprises a projection arranged to extend into the aperture.

18. A magnetic stimulation coil arrangement according to claim 1 wherein the rearward side of the one or more windings comprises a peripheral edge, and the distortion arrangement comprises a lip that extends around at least a portion of the peripheral edge.

19. A magnetic stimulation coil arrangement for use in apparatus for the magnetic stimulation of tissue, the magnetic stimulation coil arrangement comprising one or more coil windings formed from an elongate conductive element and having a forward side for presentation to a patient and a rearward side, the magnetic stimulation coil arrangement further comprising a distortion arrangement for distorting a magnetic field produced by the one or more coils positioned adjacent to the rearward side, the distortion arrangement having a plurality of ferromagnetic particles dispersed therein and a carrier for carrying the ferromagnetic particles, the ferromagnetic particles being spaced from one another by the carrier wherein the ratio volume of ferromagnetic particles to carrier material is less than 1.5:1.

20. A magnetic stimulation coil arrangement according to claim 19 wherein the ratio of the volume of ferromagnetic particles to carrier material is less than 1:1.

21. A magnetic stimulation coil arrangement according to claim 19 wherein the ferromagnetic particles comprise iron filings.

22. A magnetic stimulation coil arrangement according to claim 19 wherein the carrier is a matrix.
